(19) United States
(12) Patent Application Publication
(10) Pub. No.: US 2023/0249403 A1
(43) Pub. Date: Aug. 10, 2023

(54) Method For Producing A 3-Dimensional Object And Apparatus Therefor

(71) Applicant: Ottobock SE & Co. KGaA, Duderstadt (DE)

(72) Inventors: Mario KOPPE, WENDEHAUSEN (DE); Lars Benjamin FINKE, Landolfshausen (DE)

(21) Appl. No.: 18/004,510

(22) PCT Filed: Jul. 8, 2021
(86) PCT No.: PCT/EP2021/069004

§ 371 Date: Jan. 6, 2023

(30) Foreign Application Priority Data
Jul. 8, 2020 (DE) 10 2020 118 034.9

(51) Int. Cl. B29C 64/209; B33Y 10/00; B33Y 30/00; B33Y 80/00; B22F 10/64; B33Y 40/20; B22F 10/50; B22F 12/60; B22F 12/50; B29C 64/106; B29C 64/321; B29C 64/194

(52) U.S. Cl. CPC B29C 64/209; B33Y 10/00; B33Y 30/00; B33Y 80/00; B22F 10/64; B33Y 40/20; B22F 10/50; B22F 12/60; B22F 12/50; B29C 64/106; B29C 64/321; B29C 64/194; B22F 2998/10; B29L 2031/7532

(57) ABSTRACT

The invention relates to a method for producing a three-dimensional objects by means of an additive manufacturing process in which at least one manufacturing material is fed in a free-flowing state from at least one feed-in opening of at least one feed-in needle into a supporting material and then cured, the manufacturing material being introduced in multiple layers one after the other, wherein the feed-in needle comprises at least one tool by which at least one previous layer is machined when a current layer of the manufacturing material is introduced.

The invention relates to a method for producing a three-dimensional object by means of an additive manufacturing process in which at least one manufacturing material is fed in a free-flowing state from at least one feed-in opening of at least one feed-in needle into a supporting material and then cured, the manufacturing material being introduced in multiple layers one after the other. The invention also relates to a device for conducting such a method.

Nowadays, many types of additive manufacturing process are known from the prior art and are used to produce a wide range of three-dimensional objects. Traditionally, additive manufacturing processes are hardly suitable for producing large quantities of the respective objects, as the production of individual objects takes a lot of time. In additive manufacturing processes, especially 3D printing, the object to be produced is built up of a number of very thin layers arranged on top of each other, which are often just a few micrometers thick. As a result, the production of large objects in particular is time-consuming and has therefore not yet become established for the production of large quantities.

In the last few years, a great deal of progress has been made in this area. For example, MIT developed a three-dimensional printing process that was published in US 2018/281295 A1, for example. In this case, the object to be produced is made in a container that contains a gel suspension or another material as a support material which does not chemically react with the manufacturing material. It serves only to support the manufacturing material as long as it is not yet cured or sufficiently crosslinked. In the method, the manufacturing material is introduced in a free-flowing state, for example as a liquid or gel, at the desired positions within the support material.

The feed-in needle is used for this purpose, which can be displaced in three directions that are linearly independent of one another. Due to the density ratios between the manufacturing material and the support material, the manufacturing material that has been introduced remains in the respective position, so that three-dimensional objects can be printed especially quickly by putting the manufacturing material at the desired points and subsequently cross-linking, setting or curing it there. This process is significantly faster than previous 3D printing methods and also makes it possible to produce flexible or elastic objects. It also allows a wide range of materials to be printed that have not been specifically optimized for additive manufacturing processes, but have become established in conventional casting processes. This includes biocompatible silicones, for example. However, certain material combinations of manufacturing material and support material present problems. A similar method is described by the company NSTRMNT at http://nstmnt.com/#/suspended-depositions/ under the key term “Suspended Deposition”.

The method is used to create a three-dimensional object in different layers arranged one on top of the other. However, these layers can be made to be significantly thicker than in conventional methods, so that large objects can also be produced relatively quickly. To achieve this, however, it is necessary that different layers of the manufacturing material that are applied on top of each other not only remain at the desired position within the support material, but that a sufficiently secure bonding forms between these layers, thereby resulting in a three-dimensional object. In addition to this basic layer bonding, which is necessary to create cohesive components, it is desirable for the materials that have not yet cured to flow into each other in order to obtain object surfaces that are as smooth as possible. This is only insufficiently the case for certain material combinations, so that the determination of suitable process parameters and material combinations sometimes incurs a great deal of research effort and is therefore cost- and time-intensive.

The invention is therefore based on the task of improving a method according to the preamble in such a way that the disadvantages of the prior art are eliminated or at least reduced.

The invention solves the addressed task by way of a method according to the preamble of claim 1, characterized in that the feed-in needle comprises at least one tool by which at least one previous layer is machined when a current layer of the manufacturing material is introduced. Preferably, at least the immediately preceding layer of the manufacturing material is machined when the current layer is introduced.

The invention is based on the knowledge that, with regards to the adhesion of different layers of manufacturing material to or on each other, it is advantageous if a previously introduced layer of the manufacturing material is re-machined when the current layer is introduced, so that the contact between the different layers is preferably improved and the adhesion and bonding between the different layers thus increased and/or the surface quality enhanced. Surprisingly, it is not necessary to increase, for example, a hydrostatic pressure acting on the support material for this purpose or to increase the pressure at which the manufacturing material is introduced from the feed-in needle into the support material. These measures can be individually or collectively advantageous, but are not required for the functionality of the invention.

In the method described here, the feed-in needle is moved through the support material. It usually has a longitudinal direction that extends from one end of the feed-in needle, where the manufacturing material is introduced into the feed-in needle, to an opposite end of the feed-in needle, where the manufacturing material leaves the feed-in needle. At this end of the feed-in needle in particular, where a feed-in opening is provided, the feed-in needle can feature a bend, kink or curve, without this changing the longitudinal extension of the feed-in needle within the context of the present invention. The feed-in needle preferably has a tubular section, which is designed to be straight and defines the longitudinal extension of the feed-in needle. In this case, a feed-in needle with a longitudinal extension can be moved in directions perpendicular to the longitudinal extension. A wide range of geometric paths can be traced and the feed-in needle moved along these paths. Additionally or alternatively, however, the feed-in needle can also be moved along its longitudinal extension. This corresponds to a movement in the “z” direction, while the movement perpendicular to this corresponds to a movement in an “x”-“y” plane. Of course, movements are also possible that correspond to a combination of these directions.

A feed-in needle that does not have a straight section which could define a longitudinal direction can of course also be moved along these directions. The direction in which a feed-in needle is moved is described hereinafter as a direction of movement.

The at least one tool is preferably made of the same material as the feed-in needle, for example a metal or a plastic. However, it can also be made of a different material to the feed-in material. In a preferred embodiment, the at least one tool is a mandrel or a pin or a flat object similar to a trowel. Particularly preferably, the at least one tool protrudes beyond the end of the feed-in needle along with “z” direction, which, especially preferably, corresponds to the direction of the earth's gravitational field. Particularly preferably, the at least one tool protrudes beyond the feed-in opening of the feed-in needle in this direction.

During introduction of the current layer of manufacturing material, the at least one tool preferably projects into at least one preceding layer, at least one tool preferably being arranged in front of the feed-in needle in the direction of movement of the feed-in needle. During production of the three-dimensional object, the feed-in needle is consequently moved through the support material in such a way that a current layer of manufacturing material is introduced into the support material and, in the process, comes into contact with a previous layer, preferably the layer introduced immediately before. Advantageously, the at least one tool is arranged in front of the feed-in needle in the direction of movement of the feed-in needle. In this position, it has two effects. On the one hand, the tool splits and tears the support material in the direction of movement immediately in front of the feed-in needle, making it easier to move the feed-in needle through the support material in the direction of movement. On the other hand, the tool preferably protrudes beyond the feed-in opening, so that it at least protrudes into the layer of the manufacturing material that was introduced into the support material immediately beforehand. As a result, this layer is also machined, in particular slit, ripped or cut open.

Preferably, the at least one tool is so long and projects so far beyond the feed-in opening of the feed-in needle that several layers of the manufacturing material, which have already been introduced into the support material, are machined accordingly, i.e. in particular slit open, ripped open or cut open. In a preferred embodiment, the at least one tool has a bead similar to a ship's bow, and protrudes with this bead beyond the feed-in needle in the direction of movement of the feed-in needle. When the feed-in needle is moved, this bead is preferably displaced along a previously introduced layer of manufacturing material and moved in the layer. This causes the layer in which the bead moves to open and expand, and the current layer comes into contact over a particularly large surface area.

The current layer of the manufacturing material is then applied to the at least one layer of manufacturing machined in this way and comes into contact with it. Surprisingly, a significantly stronger bond between the various layers is achieved during subsequent curing, cross-linking or setting.

Preferably, the at least one tool mixes or smooths the current layer and the at least one preceding layer and/or presses them together. To this end, it is advantageous if the at least one tool is arranged next to the feed-in needle in the direction of movement of the feed-in needle. In this arrangement too, the at least one tool preferably protrudes beyond the feed-in opening of the feed-in needle in the “z” direction. With this embodiment, the three-dimensional object produced has a constant or at least almost constant wall thickness and/or a smooth and even surface. This also improves the contact between the individual layers and enhances the bond between the individual layers during subsequent curing, cross-linking or setting. In a preferred embodiment, the feed-in needle preferably features at least two tools, which are preferably arranged opposite each other on two different sides of the feed-in needle. As a result, both an inner surface and an outer surface of the three-dimensional object to be produced can be smoothed, thereby further strengthening the bond between the individual layers.

Advantageously, the at least one tool is arranged behind the feed-in needle in the direction of movement. It protrudes into both the current layer and into at least one preceding layer. Particularly preferably, it protrudes into multiple preceding layers. Since the at least one tool is arranged behind the feed-in needle in the direction of movement, when the feed-in needle is moved not only is the layer of manufacturing material already previously introduced into the support material slit open, ripped open or cut open or machined in any other way, but also the current introduced layer.

In a preferred embodiment, the feed-in needle has multiple tools by which the effects and machining of the different layers described above can be combined. In particular, trowel-like, flat tools that can be used to smooth one side of the three-dimensional object to be produced can have different shapes and, in particular, can extend over several of the previously introduced layers.

In a preferred embodiment, the three-dimensional object is an orthopaedic device, in particular a prosthesis liner. In the present case, an orthopaedic device is understood particularly to mean orthoses and prostheses and their components as well as exoskeletons to support body parts. Orthopaedic shoes, shoe inserts and similar devices are also considered orthopaedic devices.

A range of different materials are used in orthopaedic devices that take into account the various profiles of requirements of the respective device. Nowadays, prosthesis sockets in particular are produced for the patient on an individual basis and are therefore especially well-suited for the use of an additive manufacturing process. The prosthesis is fixed and arranged on an amputation stump. A range of fixing systems exists, one fixing system being known as vacuum socket technology. In this process, the volume between the amputation stump and the inner wall of the socket is evacuated when the device is in the mounted state. A prosthesis liner can be arranged on the stump for sealing and cushioning purposes, said prosthesis liner usually comprising a closed distal end and a proximal access opening and surrounding the stump when mounted. By inserting the stump equipped with the liner, a volume is created between the outer side of the prosthesis liner and the inner side of the prosthesis socket that can be evacuated, which leads to a force-locking connection between the socket and the liner.

To achieve a permanent attachment of the prosthesis socket, it is necessary to seal the volume between the prosthesis liner and the prosthesis socket against the atmosphere. For this purpose, so-called caps or collars are provided that are pulled over the proximal edge of the prosthesis socket and rest on the outer side of the liner or the stump, thereby sealing a gap. As an alternative, sealing lips may be arranged on the outer side of the liner or the inner side of the socket. The method of the type described here renders it possible to produce such a liner either completely or partially by means of the additive manufacturing process, wherein sealing lips and other elements arranged on the liner are preferably also produced in the same manufacturing step by means of the additive manufacturing process.

The invention also solves the addressed task by way of a device for carrying out a method described here. Such a device usually features a container that holds the support material and into which the feed-in needle protrudes. In this case, the feed-in needle can be moved in three directions, which are linearly independent from each other, and has at least one feed-in opening, which is preferably arranged at the lower end of the feed-in needle. Manufacturing material is introduced through the feed-in opening and through the feed-in needle into the container and thus into the support material. In addition, the feed-in needle has at least one tool that enables the machining of a layer of the manufacturing material already located in the support material when a current layer of manufacturing material is introduced into the support material through feed-in opening.

Preferably, the at least one tool can be positioned in front of , next to or behind the feed-in needle in the direction of movement of the feed-in needle. Particularly preferably, the at least one tool can be attached to the feed-in needle in predetermined orientations. For this purpose, the tool and the feed-in needle preferably each have a positioning element by which the orientations in which the two components can be attached in relation to one another can be fixed. For example, a projection can be provided on an outer surface of the feed-in needle and a corresponding recess provided on the tool, which ensures the orientation of the two components in relation to one another. If the feed-in needle has multiple corresponding projections and/or the tool has multiple corresponding recesses, different orientations can be achieved. Of course, the recess can also be arranged on the needle and the projection arranged on the tool.

Alternatively or additionally, the at least one tool is arranged on the feed-in needle in such a way that the orientation and/or the position of the tool on the feed-in needle can be infinitely adjusted.

The device preferably comprises at least one tool that is designed to correspond to the feed-in needle or a part of the feed-in needle.

In a preferred embodiment, the feed-in needle is arranged such that it can be rotated about its longitudinal axis, the device preferably comprising at least one drive for rotating the feed-in needle about its longitudinal axis. Such a drive may be a motor, for example, such as an electric motor. The orientation of the at least one feed-in opening of the feed-in needle relative to the direction of movement of the feed-in needle can therefore be changed. For example, the direction of movement of the feed-in needle can be changed without the orientation of the feed-in opening relative to this direction of movement changing. Alternatively, the orientation can even be changed without the direction of movement changing. Of course, it is also possible to change both the direction of movement of the feed-in needle and the orientation of the feed-in needle relative to this direction of movement.

In a preferred embodiment, the at least one tool is arranged in the lower area of the feed-in needle where the feed-in opening is also located. If the feed-in needle can be rotated via a drive about its longitudinal axis, the orientation of the at least one tool relative to the direction of movement of the feed-in needle can consequently also be changed, if desired. In the same way, it is possible to retain the orientation of the at least one tool relative to the direction of movement of the feed-in needle, even if this direction of movement changes.

Advantageously, the feed-in needle features a flow profile that orients the feed-in needle along the direction of movement of the feed-in needle. This can be as in addition or as an alternative to a drive. In this case, if the direction of movement of the feed-in needle changes, the flow profile ensures that the feed-in needle also rotates about its longitudinal axis and orients itself in such a way that the flow profile of the feed-in needle offers a minimal flow resistance in the new direction of movement.

In a preferred embodiment, the feed-in needle has at least one orientation element, which protrudes from part of the feed-in needle and rotates it about its longitudinal axis when the feed-in needle is moved. The orientation element can be part of the flow profile and act as such.

Advantageously, the orientation element and/or the at least one tool are attached to the feed-in needle such that they can be adjusted.

In the following, a number of embodiment examples of the invention will be explained in more detail with the aid of the accompanying drawings. They show

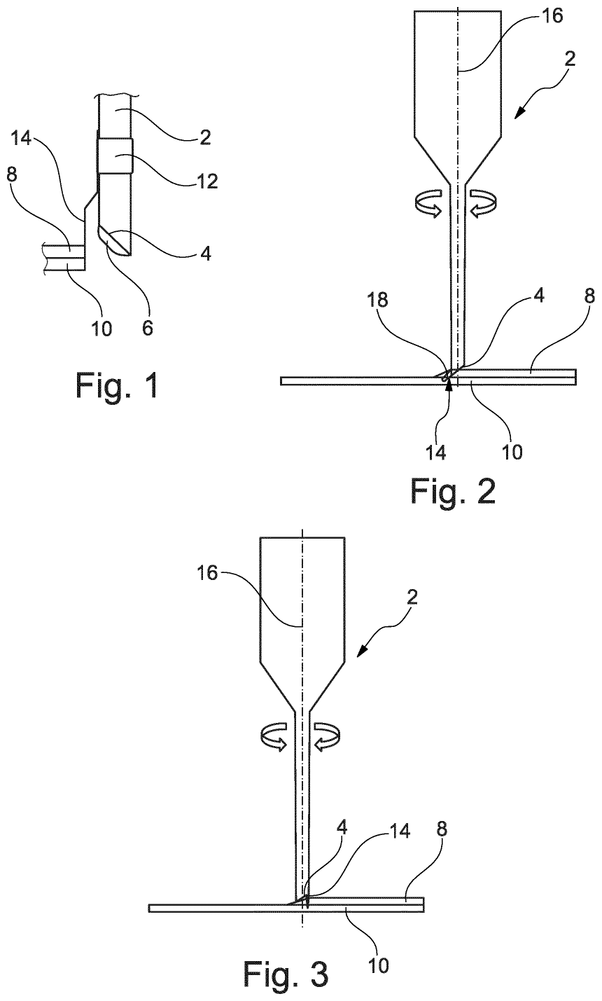

FIGS. 1 to 3—schematic representations of a part of a device according to first embodiment examples of the present invention,

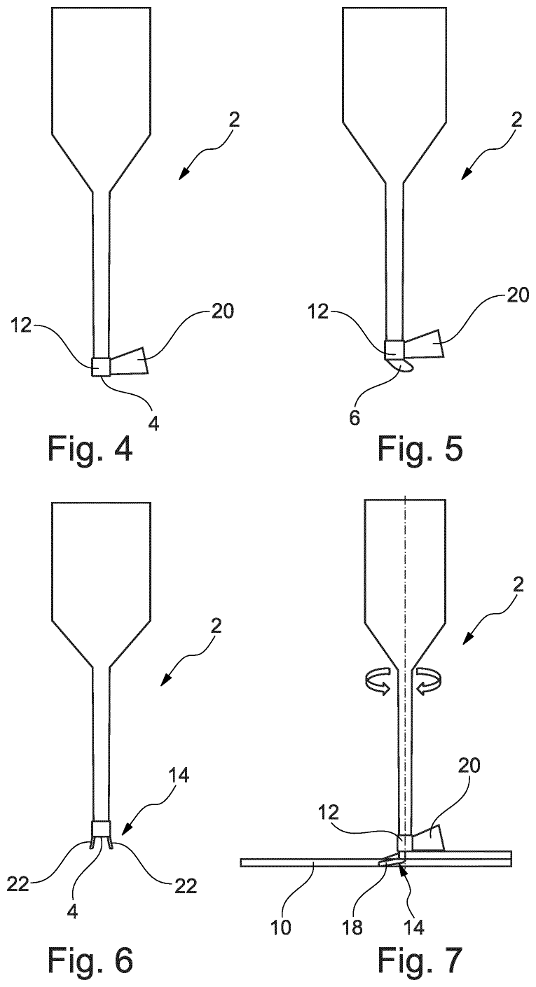

FIGS. 4 to 7—schematic representations of the part of a device according to further embodiment examples,

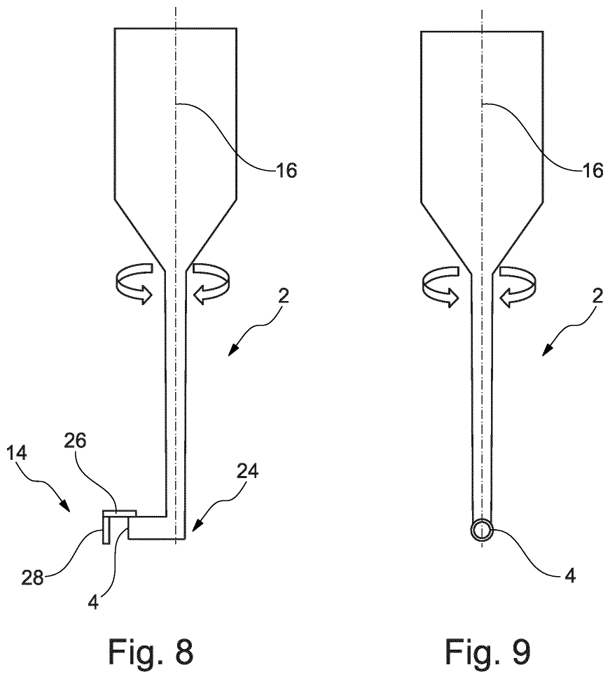

FIGS. 8 to 12—schematic representations of the part according to further embodiment examples from different perspectives,

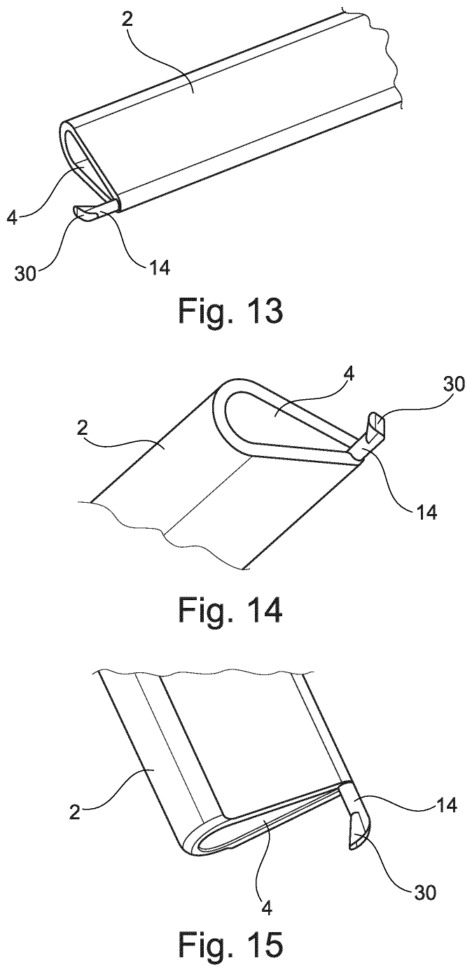

FIGS. 13 to 15—schematic representations of the part according to a further embodiment example from different perspectives,

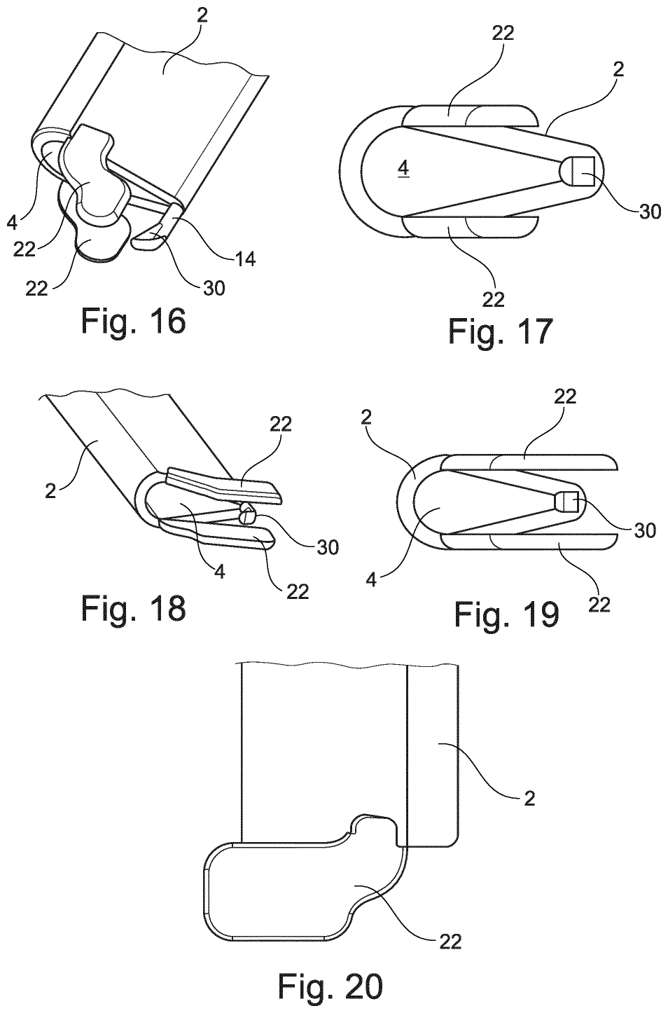

FIGS. 16 and 17—schematic representations of the part according to a further embodiment example from different perspectives,

FIGS. 18 to 20—schematic representations of the part according to a further embodiment example from different perspectives and

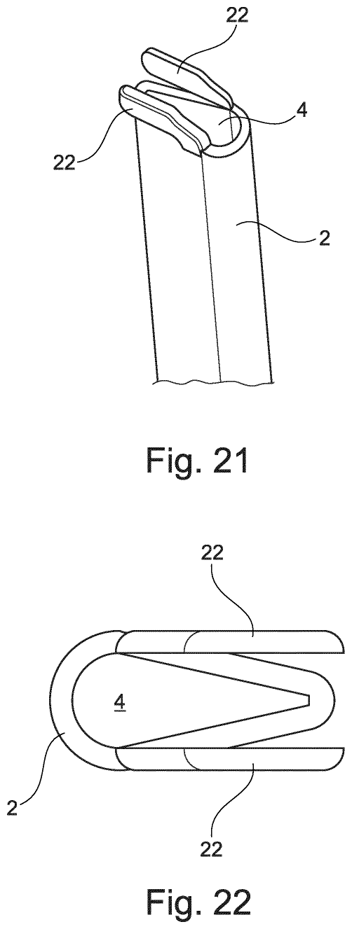

FIGS. 21 to 22—schematic representations of the part according to a further embodiment example from different perspectives.

FIG. 1 shows the end of a feed-in needle 2 that has a feed-in opening 4 at its lower end, by which the manufacturing material 6 is introduced into a support material, not depicted. During this process, a current layer 8 of the manufacturing material 6 is applied to a preceding layer 10 of the manufacturing material 6. Via a collar 12, a tool 14 in the form of a mandrel is arranged on the feed-in needle 2 in such a way that it projects beyond the feed-in opening 4 along the longitudinal direction of the feed-in needle 2, which in FIG. 1 extend from top to bottom. In the representation depicted in FIG. 1, the feed-in needle 2 is moved to the right, so that the direction of movement extends to the right. The at least one tool 14 is therefore arranged behind the feed-in needle 2 in the direction of movement. It extends through the current layer 8 and the preceding layer 10 of the manufacturing material 6, thereby machining both layers. In the present example, the two layers are ripped open.

FIG. 2 schematically shows a representation of a feed-in needle 2 that has a feed-in opening 4 at its lower end. The feed-in needle 2 is mounted such that it can rotate about its longitudinal direction 16, depicted by the dashed line: this is represented by the two arrows. A current layer 8 of the manufacturing material 6 is applied to a preceding layer 10 of the manufacturing material 6 through the feed-in opening 4. The tool 14, designed in this case as a bead 18, is located at the lower end of the feed-in needle 2. In FIG. 2, the direction of movement of the feed-in needle 2 extends to the left, so that the tool 14 in the form of the bead 18 is arranged in front of the feed-in needle 2 in the direction of movement. Therefore, the tool 14 in the form of the bead 18 only machines the previous layer 10 of the manufacturing material 6, which is indicated by a small triangle-shaped attachment on the previous layer 10.

FIG. 3 shows a different configuration of the feed-in needle 2 with its longitudinal direction 16 that can also be rotated about this longitudinal direction 16. It also has the feed-in opening 4, the tool 14 being located in this area. In this case too, the direction of movement of the feed-in needle 2 also points to the left, so that the tool 14 is arranged behind the feed-in needle 2 in the direction of movement. In FIG. 3, it protrudes downwards beyond the end of the feed-in needle 2 and particularly beyond the feed-in opening 4, thereby machining both the current layer 8 as well as the preceding layer 10 of the manufacturing material 6.

FIGS. 4 and 5 show a schematic representation of a feed-in needle 2 that comprises a orientation element 20 arranged with a sleeve 12 at the lower end of the feed-in needle 12. In contrast to FIG. 4, FIG. 5 shows that manufacturing material 6 flows out of the feed-in opening 4. The orientation element 20 changes the flow cross-section of the feed-in needle 2, which is otherwise designed to be rotationally symmetrical in relation to the longitudinal direction 16 in the example of an embodiment shown. The flow cross-section and therefore also the resistance against a movement of the feed-in needle 2 is now dependent on the direction of movement of the feed-in needle 2. If the feed-in needle 2 depicted in FIGS. 4 and 5 is moved, for example, to the left in the arrangement shown, the orientation element 20 does not cause an increase in flow resistance. However, if the feed-in needle 2 is moved perpendicular to the drawing plane in the arrangement shown, the flow resistance is significantly increased due to the orientation element 20. Since the feed-in needle 2 is designed such that it can be rotated about it longitudinal direction 16, however, the increased flow resistance caused by the orientation element 20 will cause the feed-in needle 2 to pivot about its longitudinal direction 16 until the orientation element 20 is positioned behind the feed-in needle 2 in the direction of movement.

FIG. 6 shows a representation of the feed-in needle 2 that comprises a tool 14 with two legs 22 which, in the embodiment example shown, protrude downwards beyond the feed-in opening 4 of the feed-in needle 2. The distance between the two legs 22 preferably corresponds to the width of the feed-in opening 4 and thus to the width of the introduced strand of manufacturing material 6. The two legs 22 smooth the sides of the introduced manufacturing material on both sides, thus ensuring a smoother surface of the object produced as well as improved contact between the current layer 8 and the preceding layer 10.

FIG. 7 depicts an embodiment of the feed-in needle 2 that corresponds to a combination of the representations from FIG. 2 and FIG. 4. The orientation element 20 is located at the lower end of the feed-in needle 2, said orientation element being arranged on the feed-in needle 2 by means of the collar 12. The tool 14 is arranged by way of the same collar 12, said tool being in the form of the bead 18 mentioned above. The orientation element 20 ensures that it is located behind the feed-in needle 2 in the direction of movement when the feed-in needle 2 is moved. Since the direction of extension of the bead 18, which extends to the left in FIG. 7, and the direction of extension of the orientation element 20, which extends to the right in FIG. 7, are diametrically opposite each other, it is ensured that the bead 18 is arranged in front of the feed-in needle 2 in the direction of movement. Here, the bead 18 machines the preceding layer 10 of the manufacturing material 6 onto which the current layer 8 is applied by the feed-in needle 2 from the feed-in opening 4.

FIGS. 8 and 9 depict a further embodiment of a feed-in needle 2 from different perspectives. This feed-in needle 2 can also be rotated about its longitudinal direction 16 and features a feed-in opening 4 at its lower end in FIGS. 8 and 9. In FIG. 8, it is clear that the feed-in needle 2 features a kink 24 in a lower area so that, unlike in the embodiments in the previous figures, the feed-in opening 4 is not open downwards, but rather to the left in FIG. 8. The tool 14 is arranged on the kinked part of the feed-in needle 2, said tool comprising a spacer 26 and a plate 28. The plate 28 is arranged in such a way that it is arranged in front of the feed-in opening 4 at a distance defined by the spacer 26. In FIG. 8, the plate 28 also protrudes downwards beyond the feed-in needle 2. In this configuration, by simply rotating the feed-in needle 2 about its longitudinal direction 16 it is possible to produce a circular layer of manufacturing material, the outer side of which is smoothed by the plate 28 and machined together with the outer side of a preceding layer of manufacturing material beneath it.

FIG. 9 shows the feed-in needle 2 from FIG. 8 rotated by 90°. The viewing direction now corresponds to a view into the feed-in opening 4, in front of which the plate 28, not depicted in FIG. 9, is arranged.

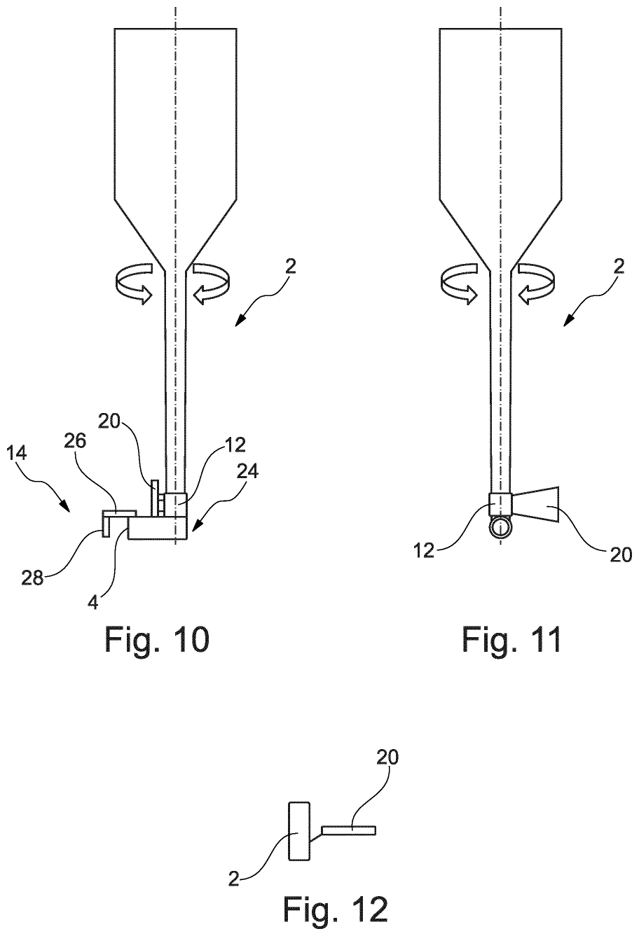

FIGS. 10 and 11 show the representations from FIGS. 8 and 9, the feed-in needle 2 now additionally having an orientation element 20. This is shown in a lateral view in FIG. 11, in which its large-area side can be seen. FIG. 10 shows the representation from FIG. 11 rotated by 90°, so that the orientation element 20 is viewed along its edge. It is fixed to the feed-in needle 2 via the collar 12. In the embodiment example shown, the kink 24 is located below the collar 12 and therefore also below the orientation element 20. The feed-in opening 4 as well as the tool 14 with the spacer 26 and plate 28 are again located at the lower end of the feed-in needle 2.

FIG. 12 depicts the kinked part of the feed-in needle 2 as well as the orientation element 20 in a view from below.

FIGS. 13 to 15 show the lower end of the feed-in needle 2 according to a further embodiment example of the present invention. The feed-in opening 4 does not have a circular cross-section; rather, the cross-section is designed in the shape of a teardrop. Due to the elongated shape of the cross-section, the feed-in needle 2 itself acts as an orientation element, ensuring that this feed-in needle 2 rotates about its longitudinal axis if the direction of movement of the feed-in needle 2 changes. The tool 14 is arranged at the pointed end of the cross-section of the feed-in needle 2 and the feed-in opening 4, which is located at the rear in the direction of movement when the feed-in needle 2 moves. In the embodiment example shown, the tool 14 has a hook 30. In the embodiment example shown, the hook 30 is pointing forwards in the direction of movement of the feed-in needle 2 and is preferably so long that it not only protrudes into the layer of the manufacturing material currently exiting the feed-in opening 4, but also into the preceding layer below.

FIGS. 16 and 17 show the end of the feed-in needle 2 according to a further embodiment example of the present invention. While FIG. 16 depicts a schematic three-dimensional view, FIG. 17 shows a view along the longitudinal axis of the feed-in needle 2 into the feed-in opening 4. In this embodiment example too, the feed-in opening 4 is designed in the shape of a teardrop, so that the shape of the feed-in needle 2 already acts as an orientation element. The tool 14 with the hook 30 is located at the rear end of the feed-in opening 4, as shown in FIGS. 13 to 15. In addition, the embodiment shown features a further tool 14 in the form of two legs 22 which project beyond the feed-in opening 4 along the longitudinal direction of the feed-in needle 2. In the embodiment example shown, these legs 22 also project beyond the hook 30. FIG. 17 shows that the legs 22 are oriented parallel to each other and do not follow the teardrop shape of the feed-in opening 4. Rather, they smooth the lateral surfaces of the current surface 8 and at least one preceding layer 10 beneath it.

Along the longitudinal direction of the feed-in opening 4, i.e. from the wide end of the feed-in opening 4 to the pointed end of the feed-in opening 4, the legs do not extend to the hook 30. Rather, they terminate before the hook 30 in this direction. This is different in the embodiment example of the feed-in needle 2 depicted in figures 18 to 20. The feed-in opening 4 is also teardrop-shaped in this embodiment example and the hook 30 is located at its pointed end. This embodiment example also features a further tool in the form of two legs 22 arranged at both sides of the feed-in needle 2 and thus at both sides of the feed-in opening 4. The legs 22 extend along the longitudinal direction of the feed-in needle 2 beyond the feed-in opening 4 and, in the embodiment example shown, also beyond the hook 30. They are oriented parallel to each other and smooth the current layer and at least one preceding layer below it, thereby resulting in a smoothest possible surface of the object to be produced. In the embodiment example depicted in FIGS. 18 to 20, the legs 22 are designed to be longer along the longitudinal direction of the feed-in opening 4 and project beyond the feed-in needle 2 and the hook 30 in the direction of movement of the feed-in needle 2. This has the advantage that the irregularities in the current layer 8 and the preceding layer 10 resulting from the hook 30 cannot cause uneven object surfaces, as the hook 30 or another tool 14 arranged at this point projects into the respective layers 8, 10, while these layers 8, 10 are located between the two legs 22.

FIGS. 21 and 22 depict a further embodiment of a feed-in needle 2. It also features the teardrop-shaped feed-in opening 4; however, in this embodiment example, there is no tool located at the pointed end of said feed-in opening. In this embodiment example, the feed-in needle 2 only features the tool in the form of the two legs 22, which project slightly beyond the feed-in needle 2 along the longitudinal direction of the feed-in opening 4.

### REFERENCE LIST

2 feed-in needle
4 feed-in opening
6 manufacturing material
8 current layer
10 preceding layer
12 collar
14 tool
16 longitudinal direction
18 bead
20 orientation element
22 legs
24 kink
26 spacer
28 plate
30 hook

1. A method for producing a three-dimensional object by an additive manufacturing process, comprising:
feeding at least one manufacturing material in a free-flowing state from at least one feed-in opening of at least one feed-in needle into a supporting material, wherein the at least one manufacturing material is introduced in multiple layers one after the other, and wherein the feed-in needle comprises at least one tool by which at least one previous layer is machined when a current layer of the manufacturing material is introduced; and
curing the at least one manufacturing material to produce the three-dimensional object.

2. The method according to claim 1, wherein during introduction of the current layer of manufacturing material, the at least one tool projects into at least one preceding layer.

3. The method according to claim 1 wherein the at least one tool mixes or smooths the current layer and at least one preceding layer and/or presses them together.

4. The method according to claim 1, wherein the at least one tool is arranged behind the feed-in needle in the direction of movement and projects into the current layer and the preceding layer.

5. The method according to claim 1, wherein the three-dimensional object is an orthopaedic device.

6. A device for carrying out a method according to claim 1.

7. The device according to claim 6, wherein the at least one tool can be arranged in front of, next to or behind the feed-in needle in the direction of movement.

8. The device according to claim 6, wherein the at least one tool is configured as a single piece with at least one part of the feed-in needle.

9. The device according to claim 6, wherein the feed-in needle is arranged such that it can be rotated about its longitudinal axis.

10. The device according to claim 9, wherein the feed-in needle features a flow profile that orients the feed-in needle along the direction of movement of the feed-in needle.

11. The device according to claim 10, wherein the feed-in needle has at least one orientation element, which protrudes from part of the feed-in needle and rotates it about its longitudinal axis when the feed-in needle is moved.

12. The device according to claim 11, wherein the orientation element and/or the at least one tool is attached to the feed-in needle such that it can be adjusted.

13. The method according to claim 2, wherein the at least one tool is arranged in front of the feed-in needle in the direction of movement of the feed-in needle.

14. The method according to claim 3, wherein the at least one tool is arranged alongside the feed-in needle in the direction of movement of the feed-in needle.

15. The method according to claim 5, wherein the orthopaedic device is a prosthesis liner.

16. The device according to claim 8, wherein the at least one tool is configured as a single piece with the feed-in needle.

17. The device according to claim 9, wherein the device comprises at least one drive for rotating the feed-in needle about its longitudinal axis.
